(19) United States
(12) Patent Application Publication
(10) Pub. No.: US 2025/0359869 A1
(43) Pub. Date: Nov. 27, 2025

(54) ASSEMBLIES INCLUDING A MEDICAL DEVICE WITH AN APPLICATOR CARD REMOVABLY COUPLED THERETO, METHODS OF CLOSING A WOUND OR INCISION USING THE ASSEMBLIES, AND A METHOD FOR PREPARING THE ASSEMBLIES

(71) Applicant: Clozex Medical, Inc., Franklin, MA (US)

(72) Inventors: Matthew M. Papagno, Franklin, MA (US); Robert Kevin Moore, Boynton Beach, FL (US)

(73) Assignee: Clozex Medical, Inc., Franklin, MA (US)

(21) Appl. No.: 19/209,262

(22) Filed: May 15, 2025

Related U.S. Application Data
(60) Provisional application No. 63/651,003, filed on May 23, 2024.

(51) Int. Cl. A61B 17/08; A61F 13/00; A61F 13/02

(52) U.S. Cl. CPC A61B 17/085; A61F 13/00059; A61F 13/024; A61F 2013/00817; A61F 2013/00846

(57) ABSTRACT

Assemblies including a medical device with an applicator card removably coupled thereto, methods of closing a wound or incision using the assemblies, and a method for preparing the assemblies are described.

### BACKGROUND

A medical device, for closing wounds or incisions, may include first and second adhesive-backed anchoring members. To close the wound or incision, the anchoring members are adhered to the patient's skin proximate to opposing edges of the wound or incision, and the anchoring members are thereafter drawn toward each other.

The medical device may be packaged with a release liner protecting adhesive-backed surfaces of the anchoring members. Once at least one anchoring member has been peeled away from the release liner, the anchoring members can be freely manipulated by the user to widen, narrow, or angle one anchoring member in relation to the other.

An experienced user may completely remove the release liner and manipulate the anchoring members in a manner that prevents their adhesive-backed surfaces from inadvertently sticking to each other or the patient's skin prior to proper application of the anchoring members to the patient's skin. Alternatively, an experienced user may remove only a first anchoring member from the release liner, thereby exposing only the adhesive-backed surface of that anchoring member, allowing the user to place the first anchoring member at a desired position with respect to a first edge of the wound or incision. Subsequently, the user may remove the remainder of the release liner from the second anchoring member and place it proximate at a desired position with respect to an opposing, second edge of the wound or incision. Thereafter, the user may draw the two anchoring members toward each other to close the wound or incision.

A new (e.g., inexperienced or first-time) user, however, may render the medical device unusable by improperly handling the medical device during or after the release liner is removed from one or both of the anchoring members. For example, the new user may inadvertently permit the exposed adhesive-backed surfaces of the anchoring members to stick to each other or, while adhering one anchoring member to the patient's skin, may inadvertently allow the opposing anchoring member to stick to the patient's skin at an undesired position with respect to the wound or incision.

### SUMMARY

A first aspect of the present disclosure relates to an assembly, comprising: a medical device comprising first and second components, wherein the first and second components are movable with respect to each other, and placeable with respect to opposing edges of a wound or incision; and an applicator card removably coupled to the medical device, wherein the removable coupling maintains positioning of the first component of the medical device with respect to the second component of the medical device.

In at least some embodiments of the first aspect, the applicator card comprises an aperture or transparent region at a central portion of the applicator card; and a size of the aperture or transparent region is configured for assessing whether opposing edges of the wound or incision will have a desired position with respect to the first and second components of the medical device while the removable coupling maintains the positioning of the first component with respect to the second component.

In at least some embodiments of the first aspect, the applicator card has indicia thereon indicating the medical device can be applied to skin of a patient while the applicator card is removably coupled to the medical device, when edges of the wound or incision, to be positioned between the first and second components of the medical device, are visible or nearly visible through the aperture or transparent region of the applicator card.

In at least some embodiments of the first aspect, the applicator card has indicia thereon indicating the medical device should be applied to skin of a patient after the applicator card has been removed from the medical device, when edges of the wound or incision, to be positioned between the first and second components of the medical device, are not visible through the aperture or transparent region of the applicator card.

In at least some embodiments of the first aspect, the applicator card has indicia thereon related to at least two ways of applying the medical device relative to the wound or incision.

In at least some embodiments of the first aspect, the indicia indicates how a user should configure the medical device while applying the medical device to skin of a patient without the applicator card coupled to the medical device.

In at least some embodiments of the first aspect, the indicia comprises one or more of indicators for aligning the medical device with respect to the wound or incision, a QR code, a uniform resource locator, natural language text, and one or more images.

In at least some embodiments of the first aspect, the QR code and uniform resource locator are associated with a video demonstrating at least one of: how the medical device is to be applied to skin of a patient with the applicator card removably coupled to the medical device; and how the medical device is to be applied to skin of the patient with the applicator card removed from the medical device.

In at least some embodiments of the first aspect, the indicia indicates the applicator card is to be removed from the medical device, prior to the medical device being applied to skin of a patient, when opposing edges of the wound or incision will not have a desired position with respect to the first and second components of the medical device if the medical device is applied to skin of a patient while the applicator card is removably coupled to the medical device and maintains the positioning of the first component with respect to the second component.

In at least some embodiments of the first aspect, the applicator card comprises one or more foldable edge portions.

In at least some embodiments of the first aspect, the one or more foldable edge portions is removably coupled to a main portion of the applicator card.

In at least some embodiments of the first aspect, the one or more foldable edge portions is removably coupled to the main portion of the applicator card using an adhesive.

In at least some embodiments of the first aspect, the applicator card is removably coupled to the medical device using an adhesive.

In at least some embodiments of the first aspect, each of the first and second components of the medical device comprises: an adhesive-backed anchoring member for adhering to skin of a patient, the adhesive-backed anchoring member comprising a wound edge for positioning proximate to the wound or incision; one or more elongated connectors extending from the wound edge of the adhesive-backed anchoring member; and one or more pulling elements joined to the one or more elongated connectors and adapted for translation of the adhesive-backed anchoring member toward the wound or incision.

In at least some embodiments of the first aspect, the applicator card is removably coupled to one or more of the adhesive-backed anchoring members, the one or more elongated connectors, and the one or more pulling elements of the first and second components of the medical device.

In at least some embodiments of the first aspect, the medical device comprises a release liner removably attached to the adhesive backing of the adhesive-backed anchoring member of both the first and second components of the medical device; and when the release liner is removed, the applicator card is the only component of the assembly maintaining the positioning of the first component with respect to the second component.

A second aspect of the present disclosure relates to a method for closing a wound or incision, comprising: providing an assembly of the first aspect; applying the medical device to skin of a patient proximate the wound or incision; after applying the medical device, removing the applicator card from the medical device; and after removing the applicator card, closing the wound or incision using the medical device.

In at least some embodiments of the second aspect, the medical device comprises a release liner removably attached to adhesive backings of adhesive-backed anchoring members of both the first and second components of the medical device, and applying the medical device comprises: removing a first portion of the release liner from the adhesive-backed anchoring member of the first component of the medical device; after removing the first portion of the release liner, adhering the adhesive-backed anchoring member of the first component to the skin of the patient proximate a first edge of the wound or incision; after adhering the adhesive-backed anchoring member of the first component, removing a second portion of the release liner from the adhesive-backed anchoring member of the second component of the medical device; and after removing the second portion of the release liner, adhering the adhesive-backed anchoring member of the second component to the skin of the patient proximate a second edge of the wound or incision.

A third aspect of the present disclosure relates to a method for closing a wound or incision, comprising: providing an assembly of the first aspect, wherein the medical device comprises a release liner removably attached to adhesive backings of adhesive-backed anchoring members of both the first and second components of the medical device; removing a first portion of the release liner from the adhesive-backed anchoring member of the first component of the medical device; after removing the first portion of the release liner, adhering the adhesive-backed anchoring member of the first component to the skin of the patient proximate a first edge of the wound or incision; after adhering the adhesive-backed anchoring member of the first component, removing the applicator card from the medical device; after removing the applicator card, removing a second portion of the release liner from the adhesive-backed anchoring member of the second component of the medical device; after removing the second portion of the release liner, adhering the adhesive-backed anchoring member of the second component to the skin of the patient proximate a second edge of the wound or incision; and after adhering the adhesive-backed anchoring member of the second component, closing the wound or incision using the medical device.

A fourth aspect of the present disclosure relates to a method for closing a wound or incision, comprising: providing an assembly of the first aspect; based on edges of the wound or incision, to be positioned between the first and second components of the medical device, not being visible through an aperture or transparent region of the applicator card, removing the applicator card from the medical device; after removing the applicator card, applying the medical device to skin of a patient proximate the wound or incision; and after applying the medical device, closing the wound or incision using the medical device.

A fifth aspect of the present disclosure relates to a method for preparing an assembly, comprising: providing a medical device of the first aspect; providing an applicator card of the first aspect; and removably coupling the applicator card to the medical device, wherein the removable coupling maintains positioning of the first component of the medical device with respect to the second component of the medical device.

### BRIEF DESCRIPTION OF DRAWINGS

For a more complete understanding of the present disclosure, reference is now made to the following description taken in conjunction with the accompanying drawings.

FIG. 1a is a top view of an example applicator card.

FIG. 1b is a bottom view of the applicator card of FIG. 1a.

FIG. 2a is a top view of another example applicator card.

FIG. 2b is a bottom view of the applicator card of FIG. 2a.

FIG. 3 is a top view of the applicator card of FIG. 1a without indicia and with foldable edge portions.

FIG. 4 is a top view of the applicator card of FIG. 1a without indicia and with foldable corner portions.

FIG. 5 is a top view of an interlaced medical device showing slit- or slice-type discontinuities in adhesive-backed anchoring members.

FIG. 6 is a bottom projection view of an interlaced medical device including release liners.

FIG. 7 is a perspective view of an interlaced medical device showing a first adhesive-backed anchoring member protected by a first release liner and a second release liner. A second adhesive-backed anchoring member is shown with a mirror image release liner configuration including a first release liner and a second release liner.

FIG. 8a is a perspective view of an example wherein a common release liner is shared between two components of a medical device.

FIG. 8b is a perspective view of an example wherein a crease is provided in a common release liner shared between two components of a medical device.

FIG. 8c is a perspective view of an example wherein a frangible line is provided in a common release liner shared between two components of a medical device.

FIG. 9 is a process flow diagram illustrating a method for closing a wound or incision.

FIG. 10 is a process flow diagram illustrating a method for closing a wound or incision.

### DETAILED DESCRIPTION

The present disclosure provides, among other things, assemblies including a medical device with an applicator card removably coupled thereto, methods of closing a wound or incision using the assemblies, and a method for preparing the assemblies.

Applicator cards of the present disclosure contain several functional and informational advantages to medical devices having at least two application methods and limited outward-facing surfaces for instructions, warnings, illustrations, steps, and other information for guiding a user of the medical device. Depending upon the design and preferred method of placement and application of the medical device, an applicator card of the present disclosure may have some or all of the features described here.

### Assemblies

Assemblies of the present disclosure include a medical device removably coupled to an applicator card. In embodiments, the medical device can take on different configurations depending on the size (e.g., distance between opposing edges) of the wound or incision to be closed.

The medical device may include at least two (i.e., first and second) opposing adhesive-backed anchoring members. The adhesive-backed anchoring members may be movable with respect to each other and designed for placement with respect to opposing edges of a wound or incision (i.e., designed for adhesion to patient skin proximate to opposing edges of a wound or incision) such that drawing the adhesive-backed anchoring members toward each other closes the wound or incision.

In embodiments of the present disclosure, the applicator card is removably coupled to the medical device in a manner that maintains a spacing/positioning of the first adhesive-backed anchoring member with respect to the second adhesive-backed anchoring member.

The medical device may be manufactured to have a release liner removably attached to both the first and second adhesive-backed anchoring members. More specifically, the release liner may be removably attached to the surfaces, of the first and second adhesive-backed anchoring members, to be adhered to the patient's skin such that the release liner protects the adhesive surfaces. In some embodiments, the assembly may be configured such that, when the release liner is removed from the medical device, the applicator card is the only assembly component maintaining the spacing/positioning of the first adhesive-backed anchoring member with respect to the second adhesive-backed anchoring member. By the applicator card being configured to maintain the spacing/positioning after the release liner is removed, the applicator card may minimize the risk of the user inadvertently sticking the adhesive-backed anchoring members to each other or, while adhering one adhesive-backed anchoring member to the patient's skin, inadvertently sticking the opposing adhesive-backed anchoring member to the patient's skin at an undesired position with respect to the wound or incision.

### Applicator Cards

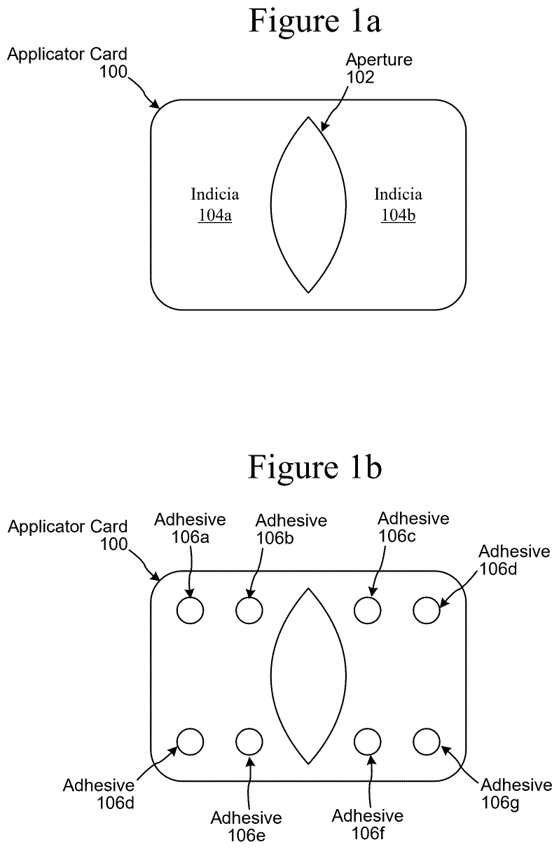

FIG. 1a-1b provides an example applicator card 100 of the present disclosure. The applicator card 100 may be constructed of a substantially or entirely opaque material. For example, the applicator card 100 may be made from one or more of opaque polyester film, opaque polyethylene film, opaque polypropylene film, densified paper/card stock, Biaxially-oriented Polypropylene (BOPP), and polyvinyl chloride (PVC). Alternatively, the applicator card 100 may be constructed from a substantially or entirely transparent material. For example, the applicator card 100 may be made from one or more of clear polyester film, clear polyethylene film, and clear polypropylene film.

Regardless of whether the applicator card 100 is constructed from one or more substantially or entirely opaque or transparent materials, the applicator card 100 may include an aperture 102. When the applicator card 100 is to be used in conjunction with a medical device including at least two adhesive-backed anchoring members to be positioned with respect to opposing edges of a wound or incision), removable coupling of the applicator card 100 to the medical device may maintain a spacing/distance between edges of the adhesive-backed anchoring members to be placed proximate to opposing edges of the wound or incision. The aperture 102 may be configured to enable a user to assess whether the spacing/distance, between edges of the adhesive-backed anchoring members to be placed proximate to opposing edges of the wound or incision, will result in the adhesive-backed anchoring members having a desired position with respect to the edges of the wound or incision if the medical device is adhered to the patient's skin while removably coupled to the applicator card 100.

In some instances, the medical device may be removably coupled to the applicator card 100 such that the spacing between the medical device's adhesive-backed anchoring members is centrally positioned in the medical device. In such instances, the aperture 102 may be centrally positioned in the applicator card 100, as illustrated in FIGS. 1a and 1b. However, the present disclosure is not limited thereto. It is envisioned that the medical device may be removably coupled to the applicator card 100 such that the spacing between the medical device's adhesive-backed anchoring members may be positioned anywhere within the medical device, and the aperture 102 can be located in the applicator card 100 at any position corresponding to that of the spacing. Put another way, the aperture 102 may be positioned anywhere within the applicator card 100, provided the aperture 102 is positioned above the spacing between the adhesive-backed anchoring members when the medical device is removably coupled to the applicator card 100.

The aperture 102 is not to be limited to any particular size and/or shape. The applicator card 100 is intended for use with wound/incision closure medical devices manufactured to have various spacings between at least two opposing adhesive-backed anchoring members to be positioned with respect to the wound/incision on the patient's skin. The size and/or shape of the aperture 102 may be configured based on the spacing between the opposing adhesive-backed anchoring members of the medical device to which the applicator card 100 is removably coupled. Generally, the aperture 102 may have a size and shape configured to enable the user to assess whether opposing edges of the wound or incision will have a desired position with respect to the “wound” edges of the opposing adhesive-backed anchoring members of the medical device while the applicator card 100 is removably coupled to the medical device and maintains the spacing/positioning of the adhesive-backed anchoring members. In some embodiments, the aperture 102 may be sized such that edges of the aperture 102 correspond to (e.g., are in line with) edges of the opposing adhesive-backed anchoring members (i.e., edges to be placed at desired positions with respect to opposing edges of the wound or incision) when the applicator card 100 is removably coupled to the medical device.

As illustrated in FIG. 1a, the top face of the applicator card 100 may include one or more indicia 104 indicating or related to when and/or how the medical device can be applied to the patient's skin while the applicator card 100 is removably coupled to the medical device. The indicia 104 may indicate the medical device can be applied to the patient's skin while the applicator card 100 is removably coupled to the medical device, when edges of the wound or incision, to be positioned between the at least first and second opposing adhesive-backed anchoring members of the medical device, are visible (or nearly visible) through the aperture 102. The indicia 104 may additionally or alternatively indicate the medical device should be applied to the patient's skin after the applicator card 100 has been removed from the medical device, when edges of the wound or incision, to be positioned between the at least first and second opposing adhesive-backed anchoring members of the medical device, are not visible through the aperture 102. In some embodiments, the indicia 104 may indicate how the user should configure (e.g., hold, place, align and/or space opposing adhesive-backed surfaces, etc.) the medical device while applying the medical device to the patient's skin without the applicator card 100 coupled thereto. In some embodiments, the indicia 104 may be related to at least two ways of applying the medical device relative to the wound or incision to be closed (i.e., with the applicator card 100 removably attached to the medical device or with the applicator card 100 removed from the medical device).

The indicia 104 may take various forms. By way of illustration and not limitation, the indicia 104 may include one or more indicators (e.g., lines, arrows, etc.) for aligning the medical device (and more particularly the at least two opposing adhesive-backed anchoring members thereof) with respect to the wound or incision, one or more quick-response (QR) codes, one or more uniform resource locators (URLs), natural language text, and/or one or more images/illustrations. In some embodiments, a QR code or URL may be associated with a webpage containing a video demonstrating how the medical device is to be applied to a patient's skin with the applicator card 100 removably coupled thereto and/or how to remove the applicator card 100 from the medical device and apply the medical device to a patient's skin with the applicator card 100 removed and/or how to decide whether to apply the medical device to the patient's skin with the applicator card attached thereto or not. In some embodiments, the indicia 104 may indicate the applicator card 100 is to be removed from the medical device, prior to the at least two opposing adhesive-backed anchoring members thereof being applied to the patient's skin, when opposing edges of the wound or incision will not have a desired position with respect to the at least two opposing adhesive-backed anchoring members of the medical device if the medical device is applied to skin of a patient while the applicator card 100 is removably coupled to the medical device and the applicator card 100 maintains the spacing between/positioning of at least the first adhesive-backed anchoring member with respect to at least the second opposing adhesive-backed anchoring member.

The applicator card 100 may have an adhesive 106, on the bottom surface thereof, for adhering the applicator card 100 to the medical device. In some embodiments, the entirety of the bottom surface of the applicator card 100 may be covered with the adhesive 106. In some embodiments, one or more discrete instances of the adhesive 106 may be applied to discrete portions of the bottom surface of the applicator card 100. The one or more instances of the adhesive 106 may be used to facilitate easier removal of the applicator card 100 from the medical device (as compared to the entirety of the bottom surface of the applicator card 100 being covered with the adhesive 106).

While FIG. 1b illustrates a particular number and placement of instances of the adhesive 106, the present disclosure is not limited thereto. The number, placement, and size of the instances of the adhesive 106 are configurable provided they are sufficient to maintain a desired adhesion to the medical device and maintain the spacing/positioning of the at least two opposing adhesive-backed anchoring members of the medical device with respect to each other. In some embodiments, the one or more instances of the adhesive 106 may be applied to one or more locations of the applicator card 100 corresponding to the at least two adhesive-backed anchoring members of the medical device. In other words, when the applicator card 100 is removably coupled to the medical device, the one or more instances of the adhesive 106 may adhere to one or more of the at least two adhesive-backed anchoring members of the medical device.

The adhesive 106 is not intended to be limited to any particular type of adhesive. By way of illustration and not limitation, the adhesive 106 may be a (low tack) acrylic pressure sensitive adhesive, (low tack) rubber-based adhesive, (low tack) silicone pressure sensitive adhesive, or combination thereof.

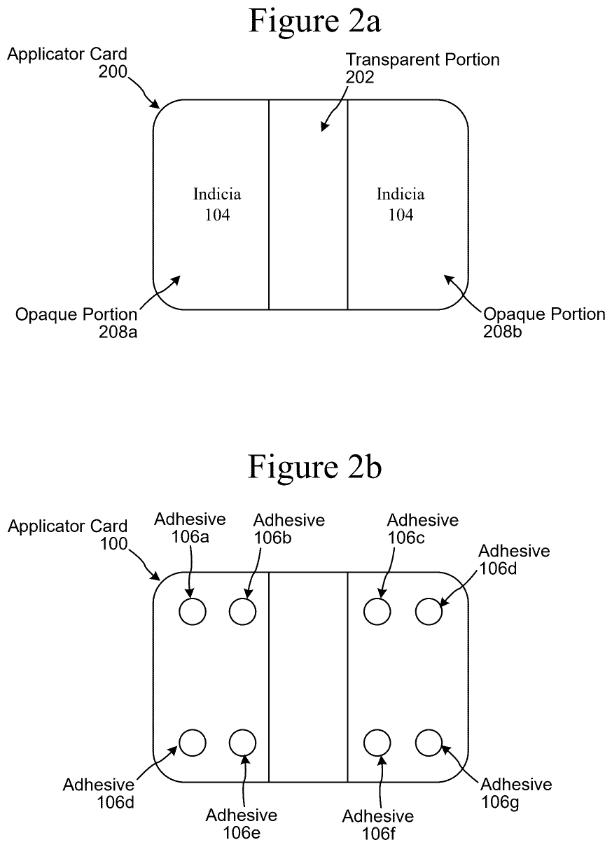

FIG. 2a-2b provides another example applicator card 200 of the present disclosure. The applicator card 200 may include one or more substantially or entirely opaque portions 208 and a substantially or entirely transparent portion 202. For example, the one or more substantially or entirely opaque portions 208 may be made from one or more of opaque polyester film, opaque polyethylene film, opaque polypropylene film, densified paper/card stock, BOPP, and PVC. For further example, the substantially or entirely transparent portion 202 may be made from one or more of clear polyester film, clear polyethylene film, and clear polypropylene film.

The substantially or entirely transparent portion 202 may be configured to enable a user to assess whether the spacing/distance, between edges of the adhesive-backed anchoring members to be placed proximate to opposing edges of the wound or incision, will result in the adhesive-backed anchoring members having a desired position with respect to the edges of the wound or incision if the medical device is adhered to the patient's skin while removably coupled to the applicator card 200.

In instances where the medical device is removably coupled to the applicator card 200 such that the spacing between the medical device's adhesive-backed anchoring members is centrally positioned in the medical device, the substantially or entirely transparent portion 202 may be centrally positioned in the applicator card 200, as illustrated in FIGS. 2a and 2b. However, the present disclosure is not limited thereto. It is envisioned that the medical device may be removably coupled to the applicator card 200 such that the spacing between the medical device's adhesive-backed anchoring members may be positioned anywhere within the medical device, and the substantially or entirely transparent portion 202 can be located in the applicator card 200 at any position corresponding to that of the spacing. Put another way, the substantially or entirely transparent portion 202 may be positioned anywhere within the applicator card 200, provided the substantially or entirely transparent portion 202 is positioned above the spacing between the adhesive-backed anchoring members when the medical device is removably coupled to the applicator card 200.

The substantially or entirely transparent portion 202 is not to be limited to any particular size and/or shape. The size and/or shape of the substantially or entirely transparent portion 202 may be configured based on the spacing between the opposing adhesive-backed anchoring members of the medical device to which the applicator card 200 is removably coupled. Generally, the substantially or entirely transparent portion 202 may have a size and shape configured to enable the user to assess whether opposing edges of the wound or incision will have a desired position with respect to the “wound” edges of the opposing adhesive-backed anchoring members of the medical device while the applicator card 200 is removably coupled to the medical device and maintains the spacing/positioning of the adhesive-backed anchoring members. In some embodiments, the substantially or entirely transparent portion 202 may be sized such that edges of the substantially or entirely transparent portion 202 correspond to (e.g., are in line with) edges of the opposing adhesive-backed anchoring members (i.e., edges to be placed at desired positions with respect to opposing edges of the wound or incision) when the applicator card 200 is removably coupled to the medical device.

As illustrated in FIG. 2a, the top face of the applicator card 200 may include the one or more indicia 104, the details of which are described herein above with respect to FIG. 1a.

Referring to FIG. 2b, the bottom surface of the applicator card 200 may also have the adhesive 106. The adhesive 106 may be applied to the applicator card 200 in one or more instances, as discussed in detail herein above with respect to FIG. 1b.

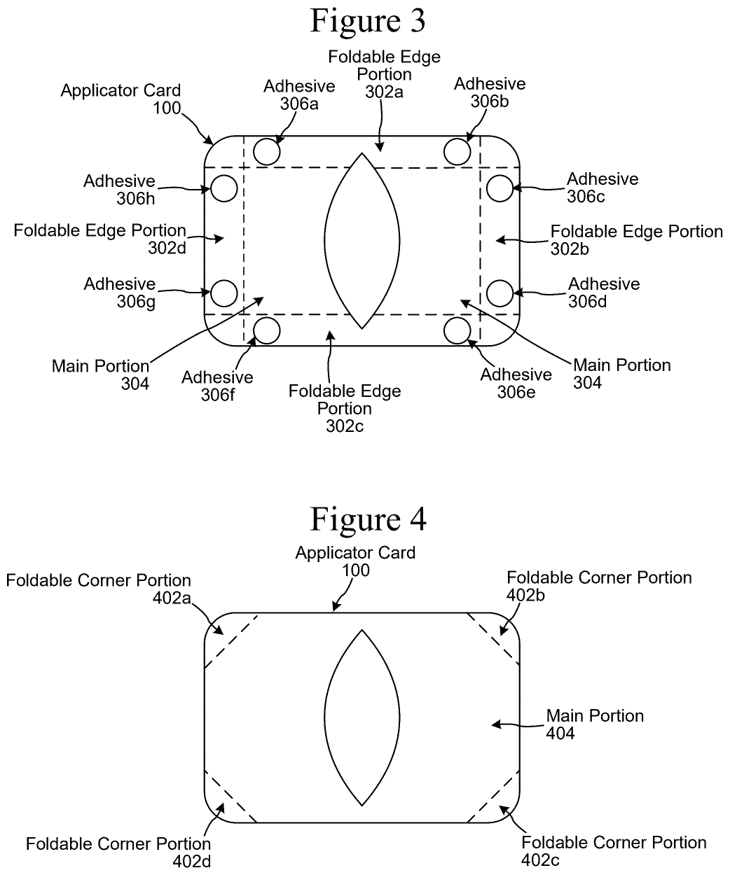

The applicator card 100/200 may include one or more foldable edge portions 302 (as illustrated in FIG. 3). While FIG. 3 illustrates the top of the applicator card 100 of FIG. 1a, the following description is equally applicable to the applicator card 200 of FIGS. 2a and 2b, or any other applicator card within the scope of the present disclosure.

As illustrated in FIG. 3, a foldable edge portion 302 may extend along an entire edge of the applicator card 100/200. The thickness of the foldable edge portion 302 is configurable. In some embodiments, a surface area of the applicator card 100/200 may be larger than the medical device to which it is removably coupled, and the applicator card 100/200 may include one or more of the foldable edge portions 302 to reduce the surface area to (about) that of the medical device. In some embodiments, the assembly (including the applicator card 100/200 removably coupled to the medical device) may be placed in packaging within which the surface area of the applicator card 100/200 does not permit it to fit. In such embodiments, the applicator card 100/200 may include one or more of the foldable edge portions 302 to reduce the surface area of the applicator card 100/200 so it can be positioned within the packaging.

The one or more foldable edge portions 302 may be removably coupled to a main portion 304 of the applicator card 100. In some embodiments, the one or more foldable edge portions 302 may be removably coupled to the main portion 304 of the top face of the applicator card 100.

The one or more foldable edge portions 302 may be removably coupled to the main portion 304 in various ways. For example, the main portion 304 may include one or more slits within which one or more corresponding portions of a foldable edge portion 302 may be inserted and thereby removably coupled to the main portion 304. For further example, a foldable edge portion 302 may have an adhesive 306 on a surface thereof, for adhering the foldable edge portion 302 to the main portion 304. In some embodiments, the entirety of the surface of the foldable edge portion 302 may be covered with the adhesive 306. In some embodiments (such as that of FIG. 3), one or more instances of the adhesive 306 may be applied to one or more portions of the surface of the foldable edge portion 302. The one or more instances of the adhesive 306 may be used to facilitate easier unfolding of the foldable edge portion 302 from the main portion 304 (as compared to the entirety of the surface of the foldable edge portion 302 being covered with the adhesive 306).

While FIG. 3 illustrates a particular number and placement of instances of the adhesive 306 on a single foldable edge portion 302, the present disclosure is not limited thereto. The number, placement, and size of the one or more instances of the adhesive 306, on a single foldable edge portion 302, are configurable provided they are sufficient to maintain a desired adhesion of the foldable edge portion 302 to the main portion 304.

However, the present disclosure is not limited to the one or more instances of the adhesive 306 being applied to a surface of the foldable edge portion 302. In some embodiments, the one or more instances of the adhesive 106 may be applied to one or more portions of the main portion 304 that abut the foldable edge portion 302 when it is folded.

The adhesive 306 is not intended to be limited to any particular type of adhesive. By way of illustration and not limitation, the adhesive 306 may be a (low tack) acrylic pressure sensitive adhesive, (low tack) rubber-based adhesive, (low tack) silicone pressure sensitive adhesive, or combination thereof.

The applicator card 100/200 may include one or more foldable corner portions 402 (as illustrated in FIG. 4). While FIG. 4 illustrates the top of the applicator card 100 of FIG. 1a, the following description is equally applicable to the applicator card 200 of FIGS. 2a and 2b, or any other applicator card within the scope of the present disclosure.

The thickness of the foldable corner portion 402 is configurable. The foldable corner portion 402 may be configured to provide a user with a “tab” to pull on and remove the applicator card 100/200 from the medical device.

The one or more foldable corner portions 402 may be removably coupled to a main portion 404 of the applicator card 100. In some embodiments, the one or more foldable corner portions 402 may be removably coupled to the main portion 404 of the top face of the applicator card 100.

The one or more foldable corner portions 402 may be removably coupled to the main portion 404 in various ways. For example, the main portion 404 may include a slit within which a corresponding portion of a foldable corner portion 402 may be inserted and thereby removably coupled to the main portion 404. For further example, a foldable corner portion 402 may have an adhesive on a surface thereof, for adhering the foldable corner portion 402 to the main portion 404. In some embodiments, the entirety of the surface of the foldable corner portion 402 may be covered with the adhesive. In some embodiments, one or more instances of the adhesive may be applied to one or more portions of the surface of the foldable corner portion 402. The one or more instances of the adhesive may be used to facilitate easier unfolding of the foldable corner portion 402 from the main portion 404 (as compared to the entirety of the surface of the foldable corner portion 402 being covered with the adhesive).

The number, placement, and size of the one or more instances of the adhesive, on a single foldable corner portion 402, are configurable provided they are sufficient to maintain a desired adhesion of the foldable corner portion 402 to the main portion 404.

However, the present disclosure is not limited to the one or more instances of the adhesive being applied to a surface of the foldable corner portion 402. In some instances, the one or more instances of the adhesive may be applied to one or more portions of the main portion 404 that abut the foldable corner portion 402 when it is folded.

In some embodiments, one or more corners of an applicator card of the present disclosure may be molded to extend out of a plane of the main portion of the applicator card, and this molded corner(s) could provide the user with one or more “tabs” to pull on and remove the applicator card from the medical device.

In instances where the applicator card 100/200 includes one or more foldable edge portions 302 and/or one or more foldable corner portions 402, the applicator card 100/200 may include the indicia 104, and the indicia may indicate how to unfold the applicator card 100/200.

The sizing and shape of an applicator card of the present disclosure is not intended to be limited to those illustrated in FIGS. 1a through 4. For example, while FIGS. 1a-4 illustrate an applicator card having rounded corners, an applicator card of the present disclosure may have right angle or other shaped corner. For further example, while FIGS. 1a-4 illustrate an applicator card having a rectangular shape, an applicator card of the present disclosure may have a non-rectangular shape.

Applicator cards of the present disclosure may be made using various materials. Generally, an applicator card of the present disclosure should have enough rigidity to support the medical device, while also having enough flexibility to allow the medical device to be placed on uneven contours of the patient.

### Medical Devices

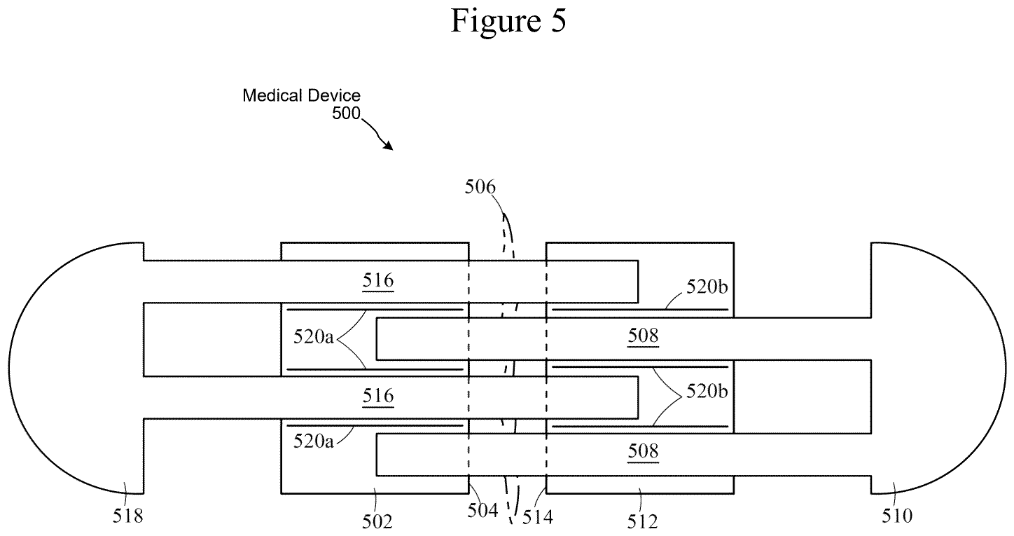

A medical device of the present disclosure may be any medical device having at least two opposing adhesive-backed anchoring members that are movable with respect to each other and designed for placement with respect to opposing edges of a wound or incision (i.e., designed for adhesion to patient skin proximate to opposing edges of a wound or incision) such that drawing the adhesive-backed anchoring members toward each other closes the wound or incision. An example of such a medical device 500 is illustrated in FIG. 5.

The medical device 500 includes a first component including a first adhesive-backed anchoring member 502 having a “wound” or “incision” edge 504 for placement at a desired location with respect to an edge of a wound or incision 506. The first component also includes one or more first elongated connectors 508 extending from the first adhesive-backed anchoring member 502 and a first pulling element 510 joined to the one or more first elongated connectors 508 and adapted for translation of the first adhesive-backed anchoring member 502 toward the wound or incision 506.

The medical device 500 also includes a second component including a second adhesive-backed anchoring member 512 having a “wound” or “incision” edge 514 for placement at a desired location with respect to another edge of the wound or incision 506. The second component also includes one or more second elongated connectors 516 extending from the second adhesive-backed anchoring member 512 and a second pulling element 518 joined to the one or more second elongated connectors 516 and adapted for translation of the second adhesive-backed anchoring member 512 toward the wound or incision 506.

The medical device 500 includes means for attaching the one or more first elongated connectors 508 to the second adhesive-backed anchoring member 512 thereby forming an attached portion and a bridging portion for each attached first elongated connector 508. The medical device 500 also includes means for attaching the one or more second elongated connectors 516 to the first adhesive-backed anchoring member 502 thereby forming an attached portion and a bridging portion for each attached second elongated connector 516.

### Interlaced and Keyhole Embodiments

Embodiments of the medical device 500 can be interlaced or non-interlaced. Interlaced embodiments are those where both the first and second components comprise two or more elongated connectors, where the first and second elongated connectors of the first component are attached to a single pulling element and the first and second elongated connectors of the second component are attached to another single pulling element. Further, an elongated connector from one of the two components passes through the void formed between the two elongated connectors of the other component. When assembled in this manner, the first and second components are linked and cannot be separated without cutting or breaking at least one of the two components. A simple analogy to such an interlaced device is a pair of interlocking rings.

One of skill in the art will recognize that more complex interlaced embodiments can be produced where, referring to a first component, interlacing extends to two or more adjacent voids formed by three or more adjacent elongated connectors. Examples of such embodiments are provided in the disclosures of U.S. Pat. Nos. 6,329,564; 6,831,205; 6,822,133; 7,511,185; 8,636,763; 7,414,168; 7,332,641; 7,354,446; 8,636,763; 7,838,718; 7,414,168; and 7,563,941, the disclosures of which are incorporated herein by reference in their entireties.

Interlaced embodiments can be produced in a variety of ways. Consider, for example, the simplest embodiment to which the analogy to interlocking rings has been made. To produce such an interlaced structure, the first and second components can be die cut as monolithic elements. One of the two components can then be cut, interlaced with the uncut component, and the cut can then be repaired, for example, with adhesive or some other appropriate means. As an alternative method for producing the simplest interlaced embodiment, one component can be die cut monolithically, and the second component can be die cut with an element, or a portion of an element missing. For example, the second component could be die cut with one of the two required elongated connectors being absent. The missing second elongated connector could be provided as a separate die cut element, complete with adhesive. Following interlacing of the first component with the partial second component, the interlaced structure could be completed by attaching the separately supplied missing elongated connector.

A non-interlaced, alternative design may be referred to as a “key-hole” design. In this design, for example, the two components of the medical device 500 are separately produced. In a preferred method, they are produced through a die cut process as monolithic components. Each of the two monolithic components comprise an adhesive-backed anchoring member, elongated connector(s), and a pulling element. The elongated connector(s) of the first component are centrally located in the assembled and applied device. The elongated connectors of the second component are spaced apart thereby creating a void, or “key-hole” through which the pulling element and elongated connector(s) of the first element are inserted. Following insertion and rotation of the first and second components into a common plane, the two components are mated in a “key-hole” arrangement and the medical device is in condition for use.

In some embodiments, the adhesive-backed anchoring members 502/512, elongated connectors 508/516, and pulling elements 510/518 are produced from a substantially inelastic polymeric material. Alternatively, they may be produced from an elastic material which is reinforced with an inelastic structural component thereby rendering the medical device substantially inelastic. For example, such inelastic materials may include monofilament polymeric line or mesh. Such reinforced polymers are referred to herein as polymeric composites. A reinforcing, inelastic structural material is referred to in the art as “scrim.” Scrim may be a woven textile or polymer, a non-woven polymer, or any other structural material that acts to stabilize the substrate. In some embodiments, the scrim reinforced substrate may have a high degree of permeability (e.g., 1,000 to 8,000 liters/sec/m²).

Additionally, non-reinforced polymers exhibiting a degree of elasticity (e.g., polyurethane or polyester) may be used in the production of the adhesive-backed anchoring members 502/512 for embodiments in which the adhesive-backed anchoring members 502/512 and elongated connectors 508/516 are produced separately, and subsequently attached to one another (i.e., non-monolithic embodiments). An example polymer for the production of the adhesive-backed anchoring members 502/512 is polyurethane having a thickness of 3-12 mils. Such a polymer is breathable and exhibits a degree of flexibility. If a non-reinforced elastic polymer is used to produce an adhesive-backed anchoring member 502/512, it may be preferable to reinforce the top surface of the wound edge 504/514 with an inelastic element so that the wound edge 504/514 remains substantially straight across the incision site during the closure process. Such an element is referred to herein as a “wound edge bar.” One skilled in the art will recognize that a wide range of inelastic polymers, or even metals, can be utilized in the production of a wound edge bar for the purpose of providing rigidity to the wound edge. Vapor permeable polymeric materials that satisfy the other requirements for use in the manufacturing of the medical device 500 may offer improved comfort. Transparent stock may be preferred so that the healing process and the entire wound site can be monitored easily. Therefore, at least the first and second adhesive-backed anchoring members 502/512 are produced from transparent stock in some embodiments. As an alternative design choice, colored or opaque stock may be used in the production of at least the first and second adhesive-backed anchoring members 502/512 when circumstances (e.g., cost considerations) dictate or permit.

### Sheet Stock

In some embodiments, the adhesive-backed anchoring members 502/512, elongated connectors 508/516, and pulling elements 510/518 are produced from sheets or rolls of polymeric material or polymeric composite material (e.g., polyurethane or polyester). The sheet or roll stock is sometimes referred to as “film” as the thickness in some embodiments ranges from about 0.5 mil to about 5 mil and may vary depending upon application. Die cutting these elements from polymeric sheet stock to provide two monolithic components (i.e., having no seams or joints) which, when assembled/packaged comprise the medical device 500, is a particularly cost-effective approach to manufacturing. Die cutting can be combined with other assembly steps, for example, in connection with the production of interlaced embodiments as discussed elsewhere herein. Laser and ultrasonic trimming devices are also examples of equipment that can be used to cut the components of the medical device 500. The sheet stock may be perforated to allow for the exchange of air with the skin beneath the medical device 500.

### Adhesives

The adhesives selected for use in connection with the medical device 500 may meet a number of requirements. First, adhesive which is to contact the patient's skin may be selected to minimize the potential for adverse reaction by the skin. That is, the adhesive selected may be hypoallergenic. Additionally, all adhesives, whether or not they are intended to contact the skin, should provide a secure hold for a period of time sufficient for the healing process to progress to the point where removal of the medical device 500 is appropriate. An adhesive hold period of about 7-10 days is generally suitable.

Adhesive is a preferred means of attaching one or more elongated connectors 508/516 to an adhesive-backed anchoring member 502/512. In one embodiment, adhesive is applied to at least a portion of the bottom surface of the elongated connectors 508/516 for attaching the elongated connectors 508/516 of one of the two components to the applied adhesive-backed anchoring member 502/512 of the other component. Alternatively or additionally, adhesive may be applied to a portion of the top surfaces of the first and second adhesive-backed anchoring members 502/512. Release liners may be used to protect applied adhesives prior to application of the medical device 500.

### Elongated Connectors

The elongated connectors 508/516 have two parts or portions, an attached portion and a bridging portion. The attached portion of the elongated connectors 508/516, as the name indicates, is that portion which is attached to the adhesive-backed anchoring member 502/512 of the opposing component following application of the medical device 500 to the patient's skin. The bridging portion is the portion of the elongated connector 508/516 which spans over the wound or incision. In some embodiments, the bottom surface of the bridging portion contains less adhesive than the attached portion. In some embodiments, the entire bridging portion is free of adhesive or, alternately, has adhesive but this is blocked with another film (kill layer) to render the adhesive in the bridging portion nonfunctional.

The dimension of the elongated connectors 508/516 may be strap-like in that their width is substantially greater than their thickness. Since the point of attachment between the first and second adhesive-backed anchoring members 502/512 is between the underside/backside of attached portions of elongated connectors 508/516 with the top surface of the adhesive-backed anchoring members 502/512, maximizing the area of contact will result in a more secure closure of the medical device 500 because the area of adhesive contact is maximized. Thus, from the standpoint of security of closure, wider attached portions are preferred. However, as the width of all the elongated connectors 508/516 is increased, the distance between elongated connectors 508/516 necessarily is decreased.

As was stated in U.S. Pat. No. 6,329,564, the disclosure of which is incorporated herein by reference: There is no absolute minimum which can be stated with respect to spacing between elongated connectors 508 or 516. Preferred ranges may be stated as a percentage of device length (i.e., the dimension of the medical device generally parallel the wound or incision). For example, a spacing of between about 5% to about 10% of the medical device length is an example of an appropriate range.

This spacing provides substantial adhesive contact between attached portions of elongated connectors 508/516 with the adhesive-backed anchoring members 502/512. When the wound or incision is closed, the bridging portions of the one or more first elongated connectors 508 and the bridging portions of the one or more second elongated connectors 516 may be aligned with one another over the closed wound or incision. In some embodiments, the average width of the bridging portions is less than the average width of the attached portions of the elongated connectors 508/516. Average width is determined by measuring from the outer perimeters of the bridging portions and the outer perimeters of the attached portions.

This difference in width in the bridging portion relative to the attached portion affords advantages. Consider, for example, a device designed for maximum security. In such a device, the elongated connectors would be placed as close as possible, while still providing for a minimum acceptable degree of adjustment range. If the bridging area were narrowed in such a device, the net effect would be an increase in exposed area over the wound or incision (which is desirable for application of medicines, removal of exudates, etc.), as well as an increase in the range of adjustment (narrowing the width of the elongated connectors in the bridging portion effectively increases the distance between adjacent bridging portions).

Considering the device discussed in the preceding paragraph, holding the width of bridging portion constant, while increasing the width of the attached portions provides for greater security as the area of adhesive contact is effectively increased. It will be recognized by one skilled in the art that hybrid configurations (i.e., devices having narrowed bridging portions and widened attached portions relative to uniform width devices) represent embodiments of the present disclosure.

The elongated connectors 508/516 may be viewed as strap-like in their dimensions. In some embodiments, a portion of the elongated connectors 508/516 is cut away to increase the unobstructed surface area over the wound or incision. This tends to facilitate drainage of exudates and application of medication. This cut-out may be best produced during the die cut process. U.S. Pat. No. 6,329,564, the disclosure of which is incorporated herein by reference, depicts cut-outs, for example, in FIG. 3. The shape of the cut-out is not critical. What is important is that the structural integrity of the elongated connectors 508/516 is not compromised by the introduction of the cut-outs.

### Pulling Elements

Embodiments of the present disclosure include pulling elements 510/518 which are attached to elongated connectors 508/516, or to extensions of elongated connectors 508/516. Extensions of elongated connectors 508/516 could themselves be considered pulling elements in embodiments in which only one elongated connector is associated with a component. By definition, the attached portion of an elongated connector 508/516 attaches to the adhesive-backed anchoring member 502/512 of another component of the medical device 500. Extensions of an elongated connector 508/516 may extend the length of the elongated connector 508/516 for ease of application, and may be generally removed following the application process. Perforations or scoring may be provided to facilitate their removal. For embodiments in which the number of elongated connectors 508/516 associated with an adhesive-backed anchoring member 502/512 is greater than one, a pulling element 510/518 may be useful for joining the elongated connectors 508/516 or extensions thereof to enable a user to easily apply a pulling force to more than one elongated connector 508/516.

Removal of the pulling elements 510/518 minimizes the footprint of the applied medical device 500. This decrease in the overall size of the medical device 500 reduces the chance that a portion of the medical device 500 may be caught, for example, on clothing or a pillow. Such an occurrence could tend to pull the applied medical device 500 away from the skin thereby causing the wound or incision to open. Minimizing the overall footprint of the applied medical device 500 also tends to provide for a more comfortable fit.

### Eversion Edges

In some embodiments, the wound edges 504/514, of the first and second adhesive-backed anchoring members 502/512, are adapted to evert (or raise) skin edges to promote wound/incision healing. It is known in the art that everting, raising, or mounding of the skin edges at the wound or incision site prevents wound inversion. One way in which this can be accomplished is to provide a bend at the wound edge 504/514. The bend may be angled or arcuate. The adhesive on the lower portion of the adhesive-backed anchoring members 502/512 is also applied to the wound edge portion. When attached to the skin this eversion edge tends to lift the edges of the skin at the point of closure contact, thereby promoting wound or incision healing.

### Coding

To minimize confusion for new users of the medical device 500, the pulling elements 510/518 and adhesive-backed anchoring members 502/512 may be coded to enable user distinction. Thus, for example, the coding may comprise an observable geometric distinction between the shape of the pulling elements 510/518 and the shape of the adhesive-backed anchoring members 502/512. In some embodiments, such coding may comprise printed indicia to enable user distinction between the components. Colors may also be used to provide this distinguishing function.

### Lateral Translation Element

In instances of the present disclosure, both the first and second adhesive-backed anchoring members 502/512 comprise one or more first elongated connectors attached to a single first pulling element. In other instances, one of the first or the second adhesive-backed anchoring members, but not both, comprise one or more lateral translation elements. In these embodiments, one of the first or second adhesive-backed anchoring components comprises one or more lateral translation elements, and the other adhesive-backed anchoring component comprises a plurality of lateral translation elements, each comprising one or more elongated connectors attached to a single pulling element.

Each lateral translation element is selected from the group consisting of: a) a single elongated connector and a single pulling element; and b) a plurality of elongated connectors and a single pulling element.

### Alternative Stock

The embodiments of the medical device 500 discussed above comprise first and second components which are monolithic in nature. That is, the first component (which includes the anchoring member 502 and one or more elongated connectors 508) is produced from a single sheet of stock material without joints or seams. The same statement applies to the second component. In an alternative embodiment, the first and second components are not monolithic in nature. This alternative embodiment is based on the recognition that the desired physical properties of the anchoring members 502/512 and the elongated connectors 508/516 are not, in every instance, identical. For example, a degree of elasticity is a desirable feature in an anchoring member 502/512 when applied, for example, to an area such as a joint. An anchoring member 502/512 produced from a film having a degree of elasticity is less likely to release prematurely than an anchoring member 502/512 produced from a substantially inelastic material when applied to such an area. Elasticity may be a property to be avoided when producing elongated connectors 508/516. Stretching of elongated connectors 508/516 may allow premature opening of a wound or incision.

In embodiments in which the first and second components are not monolithic, the anchoring members 502/512 may be produced from stock having a degree of elasticity. The elongated connectors 508/516 may be produced separately from stock which is substantially inelastic. The one or more first elongated connectors 508 are then attached (e.g., with adhesive) to the first anchoring member 502 to produce a first component. A second component is similarly constructed. As discussed elsewhere, a wound edge bar may be attached to reinforce the wound edge, particularly in embodiments wherein the sheet stock employed has a degree of elasticity.

It is not a requirement that elongated connectors and anchoring members of non-monolithic components be produced from different stock material. It may be desirable, for example, to create an overlap in a portion of the elongated connectors (e.g., the bridging portion) in order to provide for additional strength. Thus, double thickness in the bridging area may be provided by producing a monolithic anchoring member including a portion of a connecting member. A separately produced elongated connector is then attached, in an overlapping manner, to the monolithic anchoring member. This creates a first component which is double-thick in the bridging portion for additional strength and further eliminates stretching.

### Reinforcing Elements

It may be desirable to reinforce the wound edge portion of the anchoring member 502/512 with another layer of less flexible stock. This “wound edge bar” would provide better translation of the force applied by the elongated connectors 508/516 uniformly along the entire wound edge 504/514. Similarly, it may be desirable to reinforce the optional pulling element 510/518, or a portion thereof, with another layer of less flexible stock. This “pull bar” would be useful in applying uniform tension from the pulling element 510/518 to all elongated connectors 508/516, as the medical device 500 is positioned for closure. This feature would become more important in embodiments of the medical device 500 intended to close long wounds or incisions where there might be up to four or more elongated connectors 508/516 to be pulled and secured to each adhesive-backed anchoring member 502/512.

### Elastic Tension Indicators

The medical device 500 may optionally include an elastic tension indicator element. The purpose of the tension indicator element is to provide a visual indication that a desired tension has been reached while applying the medical device 500. For example, materials are known in the art which change color when a predetermined tension is applied. Similarly, other graphic representations may be used for this purpose. For example, a rectangular graphic representation may be applied to an elastic tension indicator element. As this tension indicator is stretched, the graphic representation of the rectangle stretches. This element may be designed such that the desired tension is indicated when the original rectangular representation is stretched to the point where it closely approximates a geometric square.

It may be desirable that this elastic tension indicator element be removable with the pulling elements 510/518 following application of the medical device 500. At a minimum, the elastic tension indicator element should be positioned in the medical device 500 such that when the medical device 500 is applied, it is not possible for the elastic element to continue to stretch and release the desired tension previously established.

### Transdermal Drug Delivery

The medical device 500 can be optionally adapted for transdermal drug delivery. As is known in the art, a drug is deliverable transdermally through the skin. For such an application, a drug-containing patch is secured to at least one of the adhesive-backed anchoring members 502/512 in such a way that the drug can be delivered through the skin. Given the fact that there will be no adhesive contact between the skin and the adhesive-backed anchoring member 502/512 in the area of the drug delivery patch, it may be necessary to increase the size of the anchoring member 502/512 to secure the medical device 500 in such a transdermal drug delivery embodiment. Transdermal drug delivery is well known in the art and a review of the background is not necessary to enable one of skill in the art to make and use the present disclosure.

### Embedded Infection Indicators

Embedded infection indicators can be incorporated to provide for a wound closure device that can, for example, change color as an indication of the presence of unwanted bacteria. One technology utilizes the release of a fluorescent dye from nanocapsules, the release being triggered by toxins secreted by the unwanted bacteria.

### Discontinuities in Adhesive-Backed Anchoring Members

The adhesive-backed anchoring members 502/512 may be characterized by the presence of one or more discontinuities in the polymeric film allowing for the release of sweat from beneath the adhesive-backed anchoring members 502/512. The use of an inherently breathable polymeric film (i.e., a polymeric film, that without any mechanical manipulation, allows for the exchange of air with the skin beneath the adhesive-backed anchoring members 502/512) for the production of the adhesive-backed anchoring members 502/512 may not allow for the release of sweat.

Certain mechanical manipulation of an inherently breathable polymeric film that does not allow for the release of sweat will permit sweat to pass from the skin beneath the medical device 500. The same can be said for mechanical manipulation of a polymeric film that is not inherently breathable.

The introduction of discontinuities that do allow for the release of sweat from beneath the adhesive-backed anchoring members 502/512 addresses the problem of adhesion loss. There is variability in the size, number, and distribution of active sweat glands in humans. For example, according to one expert estimate, the palm of the hand has about 370 sweat glands per cm². By comparison, the back of the hand has about 200; the forehead has about 175; the breast, abdomen, and forearm have about 155; and the leg and back have about 60-80 (all expressed in sweat glands per cm²). Given this sweat gland distribution pattern, one skilled in the art will recognize that discontinuities should be introduced broadly, across the area of the adhesive-backed anchoring members 502/512, to be most effective in addressing the problem of adhesion loss.

Discontinuities can be introduced into the polymeric material used to produce the adhesive-backed anchoring members 502/512 in a variety of ways. It is not a requirement that all discontinuities introduced into a particular adhesive-backed anchoring member be homogenous or uniform. Die cutting technology is a method for the introduction of discontinuities. Die cutting is a process involving the use of a die to shear a web or webs of low-strength materials such as polymeric sheet materials. For example, a needle or pin die could be used to introduce hundreds, or even thousands of small, round discontinuities into the first and second adhesive-backed anchoring members 502/512 through a perforation process. Perforation, as used herein, refers to a process wherein a discrete piercing element, such as a pin in an array of pins assembled on a die, penetrates a material leaving no excess material on either side (e.g., entry or exit side). Whether penetration by a particular piercing element leaves excess material on one side or another (e.g., deformation of material on the exit side) depends not solely on the piercing element, but also the material being pierced (in this case a polymeric sheet material). For example, a larger gauge piercing element, the size of a small nail, for example, may create exit deformation in some polymeric sheet stocks that would not be useful for the introduction of discontinuities consistent with the present disclosure. The shape of a piercing element need not be round. There are no geometric restrictions on the shape of a piercing element.

For the introduction of larger discontinuities, like those produced by piercing a polymeric material with a small nail, a punch process may be more appropriate. A punch, as used herein, is contrasted from a perforation by the requirement that material is removed in a punch process. For example, a punch die of a particular diameter would punch, or remove, a chad from the polymeric material. Such loose chads are removed in a variety of ways in a die cutting process including, for example, inclusion of an adhesive web to capture cut chads, or a vacuum process. Punches will tend to be visible to a user inspecting the applied medical device 500 whereas slits, slices, and perforations are practically invisible to the unaided eye.

In addition to perforation or punching, as described above, cutting (e.g., die cutting) can be used to introduce one or more slits (or slices) 520 into the adhesive-backed anchoring members 502/512. Again, in view of the sweat gland distribution discussed above, a plurality of slits 520 may be preferred.

One of skill in the art will recognize that a certain amount of routine experimentation may be required to optimize discontinuity size, shape, and distribution. Certainly, larger gauge perforations, punches, or slits will allow for the transfer of sweat from the skin beneath the medical device 500. Certain smaller gauge perforations, produced for example using a pin die as discussed above, may allow for vapor transfer but not sweat transfer. It is a matter of routine experimentation to determine discontinuity parameters that will allow for sweat transfer. Optimal discontinuity design for one specific polymer sheet backed with one specific adhesive, may not work well using a different polymer sheet and/or different adhesive. A particularly soft or gummy adhesive, for example, may function in a self-healing role by flowing in to fill perforations when such perforations are introduced with a particularly small diameter piercing element.

It will also be recognized by one of skill in the art that punch-type discontinuities will tend to remove skin contact surface area (and adhesive) from an adhesive-backed anchoring member 502/512. For this reason, larger punch-type discontinuities (e.g., paper punch size discontinuities, or larger) may not be favored, at least for applications requiring high adhesion characteristics.

For a variety of reasons, slits or slices introduced into the adhesive-backed anchoring members may be preferred. For one, like perforations, slits or slices do not remove material from the adhesive-backed anchoring members 502/512 and, therefore, the adhesive-backed anchoring members 502/512 retain their full surface area and adhesive content following the introduction of the slits or slices. Slits or slices can be straight or curvilinear and the slits or slices can be relatively long (e.g. running the length or width of an adhesive-backed anchoring member 502/512) or generally short in length. Furthermore, under flexion, a slit or slice will tend to open up. This tendency serves at least two purposes that represent advantages in the context of the medica device 500. First, the “opening up” of a slit or slice under flexion enables relative unimpeded transfer of sweat from the surface of the skin beneath the adhesive-backed anchoring member 502/512 to the external environment. Second, the “opening up” of the slit or slice tends to allow the adhesive-backed surfaces adjacent to the slit or slice to remain in good adherence with the skin. The presence of the slit or slice tends to reduce peel or shear forces that tend to result in poor adherence characteristics. When introduced, in embodiments of slices that are relatively long the effect can be the creation of a plurality of adhering subdomains in the adhesive-backed anchoring member 502/512.

In some instances, the slits 520 are introduced into each adhesive-backed anchoring member 502/512 in a direction generally perpendicular to the wound edge 504/514 of the adhesive-backed anchoring member 502/512. The slits 520 may be positioned so that they will fall between elongated connectors 508/516. Slits oriented in this way tend to allow a particular adhesive-backed anchoring member 502/512 to be viewed as a unit having a number of adhering subdomains, with the adhering subdomains being divided by the introduced slits. The adhered medical device 500 opens in an accordion-like manner under flexion. The adhering subdomains can actually separate from one another over time thereby creating independent subdomains.

Mechanical manipulation of the type described above can provide for “elastic-like” properties in non-elastic materials. Although the mechanical manipulation does not change the non-elastic characteristic of the particular material, the introduction of voids or discontinuities can allow for movement in dimensions where no movement would be possible absent the voids or discontinuities.

While the length of the slits 520 can be variable, as can the number of slits between elongated connectors 508/516, it may be beneficial that one end of the slits 520 closely approach the wound edge 504/514. It may also be beneficial that the other end of the slits 520 closely approach the edge generally opposite wound edge 504/514. The reason for the stated preference as to the length of the slits 520 is that the combined shear strength of two individual segments of polymeric material exceeds that of an otherwise identical single polymeric segment construct having equal area as compared to the two individual segments combined. The effective separation of adhesive-backed anchoring member 502/512 into individual segments (also referred to as adhering subdomains), as shown in FIG. 5 will provide for enhanced shear strength as compared to the adhesive-backed anchoring member 502/512, lacking the slits 520.

It was stated above that it may be beneficial that one end of the slits 520 closely approach the wound edge 504/514. The integrity of the wound edge 504/514 should be maintained during the application process. Therefore, prior to application, the slits 520 should not extend all the way to the wound edge 504/514, thereby severing the wound edge 504/514. In another instance of the present disclosure, the slits 520 may terminate near the wound edge 504/514, and perforations may be introduced extending the slits 520 all the way to wound edge 504/514. The perforations will not separate during the application process and the integrity of the wound edge 504/514 will be maintained. However, in the weeks following application, due to flexion and stretching, the perforations may break at the wound edge 504/514 thereby allowing adhering subdomains of the adhesive-backed anchoring member 502/512 to operate more independently.

At the edge of the adhesive-backed anchoring member 502/512 generally opposite the wound edge 504/514, the slits 520 may be extended to the edge by perforation, as discussed above in connection with perforations approaching the wound edge 504/514. Furthermore, the integrity of the edge, generally opposite wound edge 504/514, is not as critical as the integrity of wound edge 504/514. Hence, the slits 520 may be introduced in the manufacturing process to sever the edge of the adhesive-backed anchoring member 502/512 generally opposite wound edge 504/514.

### Removable Coupling of Applicator Card

With respect to the example medical device 500, the applicator card 100/200 may be removably coupled (e.g., via adhesive) to one or more of the adhesive-backed anchoring members 502/512, the one or more elongated connectors 508/516, and the one or more pulling elements 510/518.

### Release Liners

The adhesive-backed surfaces of the medical device 500 may be protected (e.g., from contamination and oxidation) by the application of release liners during the manufacturing process. In some instances, multiple release liners, or release liner systems, may be used to protect a single, uninterrupted, adhesive-backed surface. A plurality of release liners may be positioned on each adhesive-backed anchoring member 502/512. In some embodiments, a first release liner, which protects the wound edge portion of an anchoring member, is removed first during the application process. In this way, the wound edge 504/514 can be adhered to the skin while leaving a protected portion of the adhesive-backed anchoring member 502/512 which can be held without the medical device 500 adhering to the fingers of the user. Once the wound edge has been applied, the second release liner can be removed to fully secure the adhesive-backed anchoring member 502/512. Illustrative release liner stock includes paper, cardboard, or polymeric sheet stock. To minimize confusion for new users of the medical device 500, the release liners may also be coded. Release liner colors or printed indicia on the release liner are examples of coding enabling a user to readily identify the order of release liner removal.

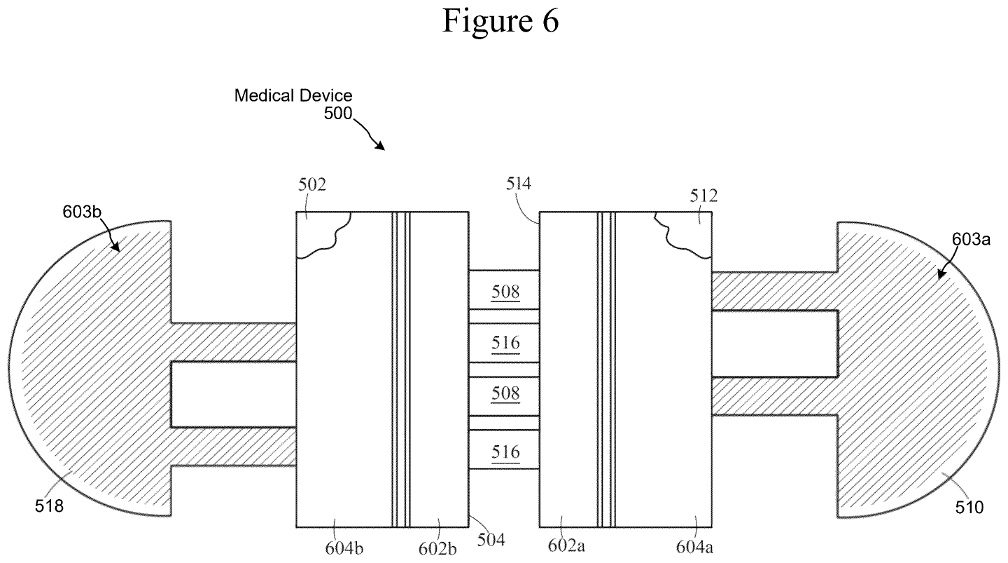

FIG. 6 is a bottom projection view of the medical device 500 showing adhesive-backed surfaces, of the adhesive-backed anchoring members 502/512, being protected by first release liners 602 and second release liners 604, and third release liners 603 (shown in cross-hatching).

The medical device may include a third release liner 603a adhered to the elongated connectors 508, with the third release liner 603a having a tab portion that extends along the first pulling element 510 and which the user may pull on to remove the third release liner 603a. Likewise, a third release liner 603b may be adhered to the elongated connectors 516, with the third release liner 603b having a tab portion that extends along the second pulling element 518 and which the user may pull on to remove the third release liner 603b.

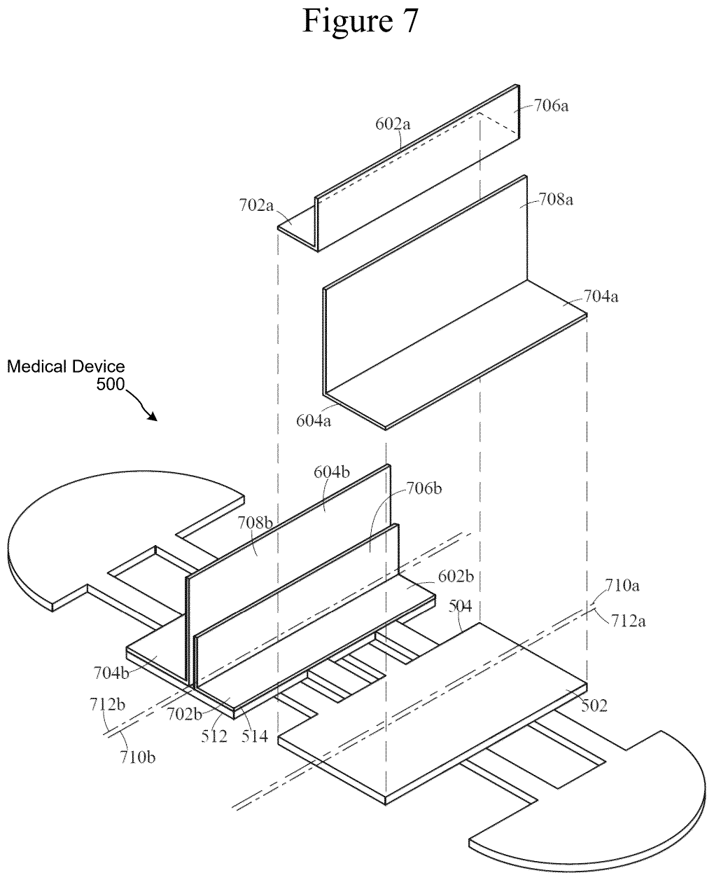

FIG. 7 is a perspective view of the medical device 500 showing the first adhesive-backed anchoring member 502 protected by the first release liner 602a and second release liner 604a. The second adhesive-backed anchoring member 512 is shown with a mirror image release liner configuration including a first release liner 602b and second release liner 604b.

Each of the release liners 602a and 604a as shown in FIG. 7 include an adhered portion (or protecting portion) in contact with and protecting an adhesive backed portion of first adhesive-backed anchoring member 502. Each of release liners 602b and 604b as shown in FIG. 7 include an adhered portion (or protecting portion) in contact with and protecting an adhesive backed portion of second adhesive-backed anchoring member 512. In FIG. 7, the adhered portion of the first release liner 602a has been assigned reference numeral 702a and the adhered portion of the second release liner 604a has been assigned reference numeral 704a. Similarly, the adhered portion of the first release liner 602b has been assigned reference numeral 702b and the adhered portion of the second release liner 604b has been assigned reference numeral 704b.

Each of the release liners shown in FIGS. 7 (602a, 604a, 602b, and 604b) also include an unadhered tab portion that extends beyond the adhesive-backed surfaces and facilitates removal of the associated release liner during application of the medical device 500. In FIG. 7, the unadhered tab portion of first release liner 602a has been assigned reference numeral 706a and the unadhered tab portion of second release liner 604a has been assigned reference numeral 708a. Similarly, the unadhered tab portion of first release liner 602b has been assigned reference numeral 706b and the unadhered tab portion of second release liner 604b has been assigned reference numeral 708b.

Tabs, or tab portions as used herein, can refer to tabs which are continuous with the associated release liner. In other words, they are produced from the same sheet stock, typically in a die cut process. Alternatively, a tab can be produced from separate stock material and attached to the release liner that it is associated with, using adhesive, for example.

As shown in FIG. 7, the first adhesive-backed anchoring member 502 has a boundary axis 710a that is parallel with the wound edge 504. Similarly, the second adhesive-backed anchoring member 512 has a boundary axis 710b that is parallel with the wound edge 514. FIG. 7 also shows that the first adhesive-backed anchoring member 502 has a boundary axis 712a that is parallel with the wound edge 504. Similarly, the second adhesive-backed anchoring member 512 has a boundary axis 712b that is parallel with the wound edge 514. Each boundary axis (710a, 710b, 712a, and 712b) defines a boundary of the adhered portion of an individual release liner. More specifically, the boundary defined by each boundary axis is an internal boundary (i.e., a boundary internal to either the first or the second adhesive-backed anchoring member). To be specific, boundary axis 710a defines an internal boundary on the first adhesive-backed anchoring member 502 that is protected by the adhered portion 702a of the first release liner 602a. Boundary axis 712a defines an internal boundary on the first adhesive-backed anchoring member 502 that is protected by adhered portion 704a of the second release liner 604a. Boundary axis 710b defines an internal boundary on the second adhesive-backed anchoring member 512 that is protected by adhered portion 702b of the first release liner 602b. Finally, boundary axis 712b defines an internal boundary on the second adhesive-backed anchoring member 512 that is protected by adhered portion 704b of the second release liner 604b.

As already mentioned, the boundary axes are parallel to a wound edge. Boundary axes 710a and 712a are parallel to the wound edge 504, and boundary axes 710b and 712b are parallel to the wound edge 514. Furthermore, because the entire adhesive-backed surface of each adhesive-backed anchoring member is completely protected by the pair of release liners, the two boundary axes associated with each adhesive-backed anchoring member must be adjacent (because the entire adhesive-backed portion of each adhesive-backed anchoring member is protected).

In use and referring to FIG. 7, the first release liners 602a and 602b may be removed and the medical device 500 subsequently applied to the skin while the applicator card 100 is removably coupled thereto. Once applied, the second release liners 604a and 604b may be removed so the remainder of the adhesive-backed anchoring members 502 and 512 can be adhered to the patient's skin.

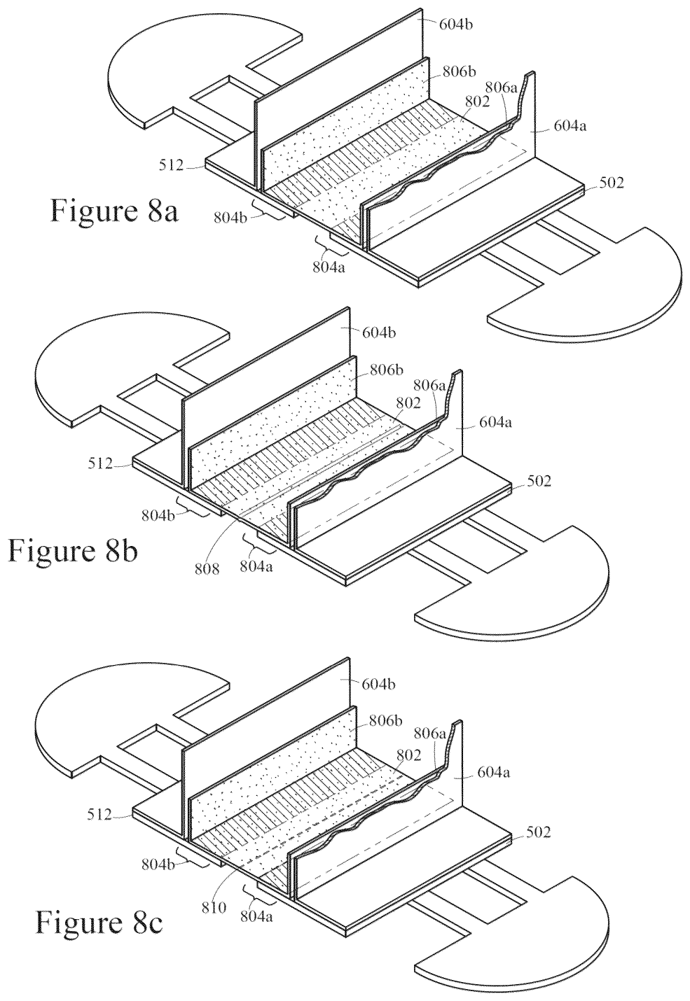

FIG. 8a shows diagrammatically an alternative instance of the present disclosure wherein a common release liner 802 (shown in stippling) is shared between the two components of the medical device 500. In particular, FIG. 8a is a diagrammatic representation showing the first adhesive-backed anchoring member 502 protected by a first adhered portion 804a (cross-hatching) of common release liner 802, and second release liner 604a. The adhesive-backed portion of second adhesive-backed anchoring member 512 is protected by a second adhered portion 804b (cross-hatching) of common release liner 802, and second release liner 604b. Common release liner 802 is provided with a first tab 806a and a second tab 806b. As depicted in the instance shown in FIG. 8a, tabs 806a and 806b are analogous to tabs 706a and 706b as discussed above in connection with FIG. 7. The transition from second adhered portions 804a and 804b of common release liner 802, to tabs 806a and 806b, respectively, are marked by a fold (not numbered). When the medical device 500 is removably coupled to the applicator card 100/200 and the common release liner 802 is removed from the medical device 500, the applicator card 100/200 may be the only component of the assembly maintaining positioning of the first and second adhesive-backed anchoring members 502/512.

In use and referring to FIG. 8a, the common release liner 802 may be removed and the medical device 500 subsequently applied to the skin while the applicator card 100 is removably coupled thereto. Once applied, the second release liners 604a and 604b may be removed so the remainder of the adhesive-backed anchoring members 502 and 512 can be adhered to the patient's skin.

Alternative instances including a common release liner 802 divided into two zones are shown diagrammatically in FIGS. 8b and 8c. The purpose of introducing two zones into the two-zone instances is to enable a user to more easily apply the medical device 500 with the applicator card 100/200 removed therefrom.

Referring to FIG. 8b, a crease 808 is provided in common release liner 802. Following removal of first adhered portion 804a from first adhesive-backed anchoring member 502, common release liner folds, or is folded along crease 808. Crease 808 provides a convenient feature for a user to “fold away” the loose tail of common release liner 802 while applying the adhesive portion of first adhesive-backed anchoring member 502 exposed by removal of first adhered portion 804a. Depending upon the depth of crease 808, the feature may enable a user to tear off a portion of the loose tail of common release liner along the crease, rather than merely folding it back. Crease 808 is located in a central region of common release liner 802 located between adhered portions 804a and 804b.

Referring to FIG. 8c, a frangible line 810 is provided in common release liner 802. Following removal of first adhered portion 804a from first adhesive-backed anchoring member 502, the loose tail portion is removed by fracturing common release liner 802 along frangible line 810. Frangible line 810 provides a convenient feature enabling a user to remove the loose tail of common release liner 802 while applying the adhesive portion of first adhesive-backed anchoring member 502 exposed by removal of first adhered portion 804a. Frangible line 810 is located in a central region of common release liner 802 located between adhered portions 804a and 804b.

### Methods of Closing a Wound or Incision

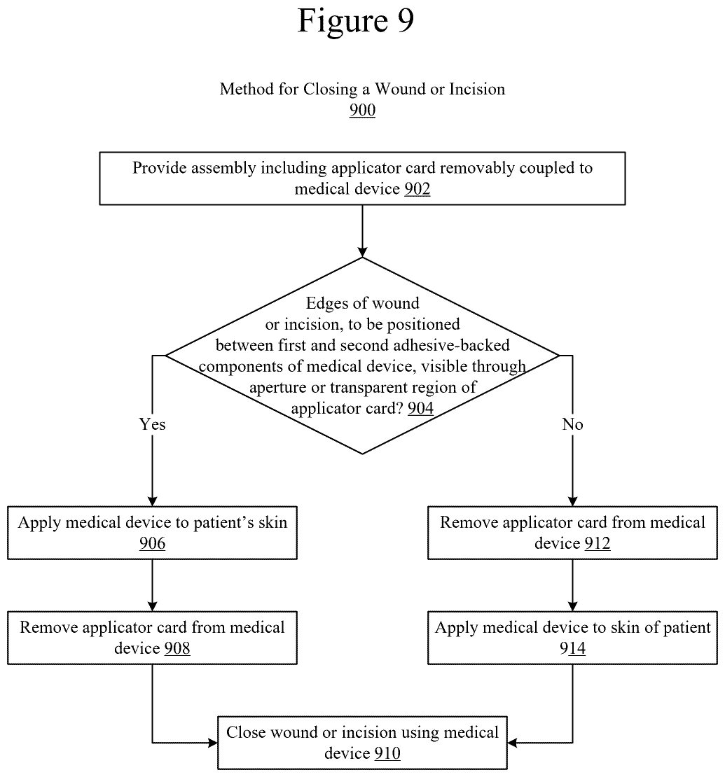

The present disclosure also provides methods for closing wounds and incisions. An example method 900 is illustrated in FIG. 9.

The method 900 includes providing (step 902) an assembly including an applicator card of the present disclosure (e.g., the applicator card 100 or 200) removably coupled to a medical device (e.g., the medical device 500) of the present disclosure.

After providing the assembly, the method includes determining (step 904) whether edges of the wound or incision, to be positioned between first and second adhesive-backed components of the medical device, are visible through an aperture or transparent region of the applicator card.

If the answer is yes, the method 900 may proceed with applying (step 906) the medical device to the patient's skin. In the event the medical device has one or more release liners, applying the medical device may include removing one or more of the release liner(s) from the medical device prior to application. For example, applying the medical device may include: removing a first portion of a common release liner a first adhesive-backed anchoring member of the medical device; after removing the first portion of the common release liner, adhering the first adhesive-backed anchoring member to the patient's skin proximate a first edge of the wound or incision; after adhering the first adhesive-backed anchoring member, removing a second portion of the common release liner from the second adhesive-backed anchoring member of the medical device; and after removing the second portion of the common release liner, adhering the second adhesive-backed anchoring member to the patient's skin proximate a second edge of the wound or incision.

After the medical device is applied to the patient's skin at step 906, the method 900 may include removing (step 908) the applicator card from the medical device and thereafter closing (step 910) the wound or incision using the medical device.

Conversely, if the answer is no at step 904, the method 900 may proceed with removing (step 912) the applicator card from the medical device and, after such remove, applying (step 914) the medical device to the patient's skin. Thereafter, the method 900 may proceed with closing (step 910) the wound or incision using the medical device.

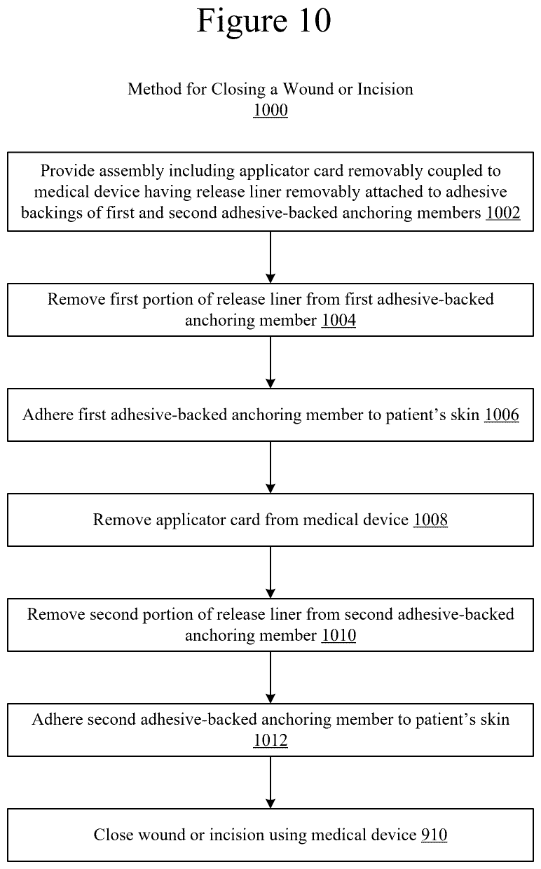

Another example method 1000 for closing a wound or incision is illustrated in FIG. 10.

The method 1000 includes providing (step 1002) an assembly including an applicator card of the present disclosure (e.g., the applicator card 100 or 200) removably coupled to a medical device (e.g., the medical device 500) of the present disclosure, with the medical device including a release liner removably attached to the adhesive backings of first and second adhesive-backed anchoring members of the medical device.

After the assembly is provided, the method 1000 includes removing (step 1004) a first portion of the release liner from the first adhesive-backed anchoring member and adhering (step 1006) the first adhesive-backed anchoring member to the patient's skin proximate a first edge of the wound or incision.

After adhering the first adhesive-backed anchoring member, the method 1000 includes removing (step 1008) the applicator card from the medical device, thereafter removing (step 1010) a second portion of the release liner from the second adhesive-backed anchoring member of the medical device, and thereafter adhering (step 1012) the second adhesive-backed anchoring member to the patient's skin proximate a second edge of the wound or incision.

After adhering the second adhesive-backed anchoring member, the method 1000 may proceed with closing (step 910) the wound or incision using the medical device.

### Further Definitions and Construction of Terms

The titles, headings, and subheadings provided herein should not be interpreted as limiting the various aspects of the disclosure. Accordingly, the terms defined herein are more fully defined by reference to the specification in its entirety. All references cited herein are incorporated by reference in their entirety.

Unless otherwise defined, scientific and technical terms used herein shall have the meanings that are commonly understood by those of ordinary skill in the art. Further, unless otherwise required by context, singular terms shall include pluralities, and plural terms shall include the singular.

In this application, the use of “or” means “and/or” unless stated otherwise. In the context of a multiple dependent claim, the use of “or” refers back to more than one preceding independent or dependent claim in the alternative only.

It is further noted that, as used in this specification and the appended claims, the singular forms “a,” “an,” and “the,” and any singular use of any word, include plural referents unless expressly and unequivocally limited to one referent.

As used herein, the term “about,” means approximately. When the term “about” is used in conjunction with a numerical range, it modifies that range by extending the boundaries above and below the numerical values set forth. Illustratively, the use of the term “about” indicates that values slightly outside the cited values (i.e., plus or minus 0.1% to 10%), which are also effective and safe are included in the value. Numerical ranges recited herein by endpoints include all numbers and fractions subsumed within that range (e.g. 1 to 5 includes 1, 1.5, 2, 2.75, 3, 3.90, 4, and 5).

As used herein, the terms “comprising” (and any form of comprising, such as “comprise,” “comprises,” and “comprised”), “having” (and any form of having, such as “have” and “has”), “including” (and any form of including, such as “includes” and “include”), and “containing” (and any form of containing, such as “contains” and “contain”) are inclusive or open-ended and do not exclude additional, un-recited elements or method steps. Additionally, a term that is used in conjunction with the term “comprising” is also understood to be able to be used in conjunction with the term “consisting of” or “consisting essentially of.”

Method steps described in this disclosure can be performed in any order unless otherwise indicated or otherwise clearly contradicted by context.

For the avoidance of doubt, insofar as is practicable any embodiment of a given aspect of the present disclosure may occur in combination with any other embodiment of the same aspect of the present disclosure. In addition, insofar as is practicable it is to be understood that any preferred or optional embodiment of any aspect of the present disclosure should also be considered as a preferred or optional embodiment of any other aspect of the present disclosure.

What is claimed is:

1. An assembly, comprising:
a medical device comprising first and second components, wherein the first and second components are movable with respect to each other, and placeable with respect to opposing edges of a wound or incision; and
an applicator card removably coupled to the medical device, wherein the removable coupling maintains positioning of the first component of the medical device with respect to the second component of the medical device.

2. The assembly of claim 1, wherein:
the applicator card comprises an aperture or transparent region at a central portion of the applicator card; and
a size of the aperture or transparent region is configured for assessing whether opposing edges of the wound or incision will have a desired position with respect to the first and second components of the medical device while the removable coupling maintains the positioning of the first component with respect to the second component.

3. The assembly of claim 2, wherein the applicator card has indicia thereon indicating the medical device can be applied to skin of a patient while the applicator card is removably coupled to the medical device, when edges of the wound or incision, to be positioned between the first and second components of the medical device, are visible or nearly visible through the aperture or transparent region of the applicator card.

4. The assembly of claim 2, wherein the applicator card has indicia thereon indicating the medical device should be applied to skin of a patient after the applicator card has been removed from the medical device, when edges of the wound or incision, to be positioned between the first and second components of the medical device, are not visible through the aperture or transparent region of the applicator card.

5. The assembly of claim 1, wherein the applicator card has indicia thereon related to at least two ways of applying the medical device relative to the wound or incision.

6. The assembly of claim 5, wherein the indicia indicates how a user should configure the medical device while applying the medical device to skin of a patient without the applicator card coupled to the medical device.

7. The assembly of claim 5, wherein the indicia comprises one or more of indicators for aligning the medical device with respect to the wound or incision, a QR code, a uniform resource locator, natural language text, and one or more images.

8. The assembly of claim 7, wherein the QR code and uniform resource locator are associated with a video demonstrating at least one of:
how the medical device is to be applied to skin of a patient with the applicator card removably coupled to the medical device; and
how the medical device is to be applied to skin of the patient with the applicator card removed from the medical device.

9. The assembly of claim 5, wherein the indicia indicates the applicator card is to be removed from the medical device, prior to the medical device being applied to skin of a patient, when opposing edges of the wound or incision will not have a desired position with respect to the first and second components of the medical device if the medical device is applied to skin of a patient while the applicator card is removably coupled to the medical device and maintains the positioning of the first component with respect to the second component.

10. The assembly of claim 1, wherein the applicator card comprises one or more foldable edge portions.

11. The assembly of claim 10, wherein the one or more foldable edge portions is removably coupled to a main portion of the applicator card.

12. The assembly of claim 11, wherein the one or more foldable edge portions is removably coupled to the main portion of the applicator card using an adhesive.

13. The assembly of claim 1, wherein the applicator card is removably coupled to the medical device using an adhesive.

14. The assembly of claim 1, wherein each of the first and second components of the medical device comprises:
an adhesive-backed anchoring member for adhering to skin of a patient, the adhesive-backed anchoring member comprising a wound edge for positioning proximate to the wound or incision;
one or more elongated connectors extending from the wound edge of the adhesive-backed anchoring member; and
one or more pulling elements joined to the one or more elongated connectors and adapted for translation of the adhesive-backed anchoring member toward the wound or incision.

15. The assembly of claim 14, wherein the applicator card is removably coupled to one or more of the adhesive-backed anchoring members, the one or more elongated connectors, and the one or more pulling elements of the first and second components of the medical device.

16. The assembly of claim 14, wherein:
the medical device comprises a release liner removably attached to the adhesive backing of the adhesive-backed anchoring member of both the first and second components of the medical device; and
when the release liner is removed, the applicator card is the only component of the assembly maintaining the positioning of the first component with respect to the second component.

17. A method for closing a wound or incision, comprising:
providing the assembly of claim 1;
applying the medical device to skin of a patient proximate the wound or incision;
after applying the medical device, removing the applicator card from the medical device; and
after removing the applicator card, closing the wound or incision using the medical device.

18. The method of claim 17, wherein the medical device comprises a release liner removably attached to adhesive backings of adhesive-backed anchoring members of both the first and second components of the medical device, and applying the medical device comprises:
removing a first portion of the release liner from the adhesive-backed anchoring member of the first component of the medical device;
after removing the first portion of the release liner, adhering the adhesive-backed anchoring member of the first component to the skin of the patient proximate a first edge of the wound or incision;
after adhering the adhesive-backed anchoring member of the first component, removing a second portion of the release liner from the adhesive-backed anchoring member of the second component of the medical device; and
after removing the second portion of the release liner, adhering the adhesive-backed anchoring member of the second component to the skin of the patient proximate a second edge of the wound or incision.

19. A method for closing a wound or incision, comprising:
providing the assembly of claim 1, wherein the medical device comprises a release liner removably attached to adhesive backings of adhesive-backed anchoring members of both the first and second components of the medical device;
removing a first portion of the release liner from the adhesive-backed anchoring member of the first component of the medical device;
after removing the first portion of the release liner, adhering the adhesive-backed anchoring member of the first component to the skin of the patient proximate a first edge of the wound or incision;
after adhering the adhesive-backed anchoring member of the first component, removing the applicator card from the medical device;
after removing the applicator card, removing a second portion of the release liner from the adhesive-backed anchoring member of the second component of the medical device;
after removing the second portion of the release liner, adhering the adhesive-backed anchoring member of the second component to the skin of the patient proximate a second edge of the wound or incision; and
after adhering the adhesive-backed anchoring member of the second component, closing the wound or incision using the medical device.

20. A method for closing a wound or incision, comprising:
providing the assembly of claim 1;
based on edges of the wound or incision, to be positioned between the first and second components of the medical device, not being visible through an aperture or transparent region of the applicator card, removing the applicator card from the medical device;
after removing the applicator card, applying the medical device to skin of a patient proximate the wound or incision; and
after applying the medical device, closing the wound or incision using the medical device.

21. A method for preparing an assembly, comprising:
providing the medical device and the applicator card of claim 1; and
removably coupling the applicator card to the medical device, wherein the removable coupling maintains positioning of the first component of the medical device with respect to the second component of the medical device.
